(19) United States
(12) Patent Application Publication
(10) Pub. No.: US 2024/0188982 A1
(43) Pub. Date: Jun. 13, 2024

(54) VALVULOPLASTY BALLOON CATHETER

(71) Applicant: MERIL LIFE SCIENCES PVT LTD., Vapi (IN)

(72) Inventors: PRAMOD KUMAR MINOCHA, Vapi (IN); DEVESHKUMAR MAHENDRALAL KOTHWALA, Surat (IN); ARPIT PRADIPKUMAR DAVE, Silvassa (IN)

(73) Assignee: MERIL LIFE SCIENCES PVT LTD., Vapi (IN)

(21) Appl. No.: 18/557,869

(22) PCT Filed: Apr. 12, 2022
(86) PCT No.: PCT/IN22/50352

§ 371 Date: Oct. 27, 2023

(30) Foreign Application Priority Data
May 3, 2020 (IN) 202021018863

(51) Int. Cl. A61B 17/3207; A61B 17/00; A61M 25/10

(52) U.S. Cl. CPC A61B 17/320725; A61B 2017/00783; A61M 25/10

(57) ABSTRACT

A valvuloplasty balloon catheter (100) is disclosed. The valvuloplasty balloon catheter (100) comprises of an inflatable member (40) having anchor shaped ends and a tubular member (50). The tubular member (50) comprises of a proximal section (50a), a distal section (50b) and a middle section (50c). The middle section (50c) includes a proximal middle portion (50c1), a distal middle portion (50c2) and an intermediate portion (50c3). The proximal middle portion (50c1) and the distal middle portion (50c2) includes a plurality of closed cells (54). At least one of the proximal section (50a) or the distal section (50b) includes a plurality of first struts (52) being anchor shaped. The plurality of closed cells (54) includes one or more zig-zag elements being placed over tapered end of the inflatable member (40) thereby allowing uniform and smooth expansion of the tubular member (50). The intermediate portion 50c3 includes a plurality of s-shaped links.

### FIELD OF INVENTION

The present invention relates to a catheter for medical devices. More specifically, the present invention relates to a valvuloplasty balloon catheter.

### BACKGROUND

Stenosis of an aortic or other cardiac valve occurs when a valve annulus narrows restricting the flow of blood through the valve when open. In order to treat such condition of valves, valvuloplasty proves to be a promising therapeutic procedure.

Valvuloplasty corresponds to widening of a stenotic valve using a balloon catheter. In valvuloplasty procedure, an operator inserts a hollow tube (catheter) into a blood vessel in the arm/groin of a patient and advances it through the aorta into the heart. Once the catheter reaches the treatment site, a balloon is expanded until the native leaflets of the valve are pushed open. Once the valve is open, the balloon as well as the catheter is removed.

However, treating blood vessels using valvuloplasty is associated with multiple shortcomings. One of the major obstacles is re-stenosis i.e. reoccurrence of stenosis.

Further, lack of effectiveness of pre-dilation in few patients has been observed. This is due to the fact that the radial pressure applied by the balloon is not always directed symmetrically, and the balloon can often slip from their original placement within the valve annulus. Both these circumstances limit the effectiveness of conventional valvuloplasty therapy.

Recently, scoring elements have been introduced for overcoming the above problems effectively. These are stent like structures mounted over the balloon which expand along with the balloon to remove calcified tissues from the implantation site at the time of pre-dilation. However, the conventional scoring elements are not bonded to the catheter properly and have reduced flexibility leading to greater force requirement when advance through torturous anatomy. Moreover, the conventional scoring elements may impart injury and trauma to the vessel wall during delivering, retraction and expansion in the vessel. Further, the existing structures of the scoring elements detach easily after multiple cycles of balloon inflation/deflation.

Therefore, there arises a requirement of a delivery device for an implant which overcomes the aforementioned challenges associated with the conventional delivery systems.

### SUMMARY

The present invention relates to a valvuloplasty balloon catheter. The said catheter includes an inflatable member having anchor shaped ends. A tubular member is mounted over the inflatable member. The tubular member includes a proximal end and a distal end. The tubular member includes a proximal section which is disposed towards the proximal end, a distal section which is disposed towards the distal end and a middle section disposed there between. At least one of the proximal section or the distal section includes a plurality of first struts, which are anchor shaped. The middle section includes a proximal middle portion, a distal middle portion, and an intermediate portion connecting the proximal middle portion and the distal middle portion. The intermediate portion includes a plurality of s-shaped links.

The foregoing features and other features as well as the advantages of the invention will become more apparent from the following detailed description, which proceeds with reference to the accompanying figures.

### BRIEF DESCRIPTION OF DRAWINGS

The summary above, as well as the following detailed description of illustrative embodiments, is better understood when read in conjunction with the apportioned drawings. For the purpose of illustrating the present disclosure, exemplary constructions of the disclosure are shown in the drawings. However, the disclosure is not limited to specific methods and instrumentalities disclosed herein. Moreover, those in the art will understand that the drawings are not to scale.

FIG. 1 depicts a balloon catheter 100 in accordance with an embodiment of the present invention.

FIG. 1a depicts the balloon catheter 100 provided with an embolic filter 200 in accordance with an embodiment of the present invention.

FIG. 2 depicts the tubular member 50 in accordance with an embodiment of the present invention.

FIG. 2a depicts the proximal section/distal section 50a/50b of the tubular member 50 in accordance with an embodiment of the present invention.

FIG. 2b depicts an alternate embodiment of the proximal section/distal section 50a/50b of the tubular member 50 in accordance with an embodiment of the present invention.

FIG. 2c depicts yet another embodiment of the proximal section/distal section 50a/50b of the tubular member 50 in accordance with an embodiment of the present invention.

FIG. 2c1 depicts yet another embodiment of the proximal section/distal section 50a/50b of the tubular member 50 in accordance with an embodiment of the present invention.

FIG. 2d depicts the couplers 60a, 60b being attached with the crimped tubular member 50 in accordance with an embodiment of the present invention.

FIG. 2e depicts the middle section 50c of the tubular member 50 in expanded state in accordance with an embodiment of the present invention.

FIG. 2f depicts the closed cell 54 of the tubular member 50 in accordance with an embodiment of the present invention.

FIG. 2g depicts the marker links 56 of the tubular member 50 in accordance with an embodiment of the present invention.

FIG. 2g1 depicts s-shaped link of the intermediate portion 50c3 in accordance with an embodiment of the present invention.

FIG. 2h depicts the columns of the tubular member 50 in accordance with an embodiment of the present invention.

FIG. 3a depicts a force vs distance graph in case of straight link in accordance with an embodiment of the present invention.

FIG. 3b depicts a force vs distance graph in case of s-shaped link in accordance with an embodiment of the present invention.

### DETAILED DESCRIPTION OF THE DRAWINGS

Prior to describing the invention in detail, definitions of certain words or phrases used throughout this patent document will be defined: the terms “include” and “comprise”, as well as derivatives thereof, mean inclusion without limitation; the term “or” is inclusive, meaning and/or; the phrases “coupled with” and “associated therewith”, as well as derivatives thereof, may mean to include, be included within, interconnect with, contain, be contained within, connect to or with, couple to or with, be communicable with, cooperate with, interleave, juxtapose, be proximate to, be bound to or with, have a property of, or the like; Definitions of certain words and phrases are provided throughout this patent document, and those of ordinary skill in the art will understand that such definitions apply in many, if not most, instances to prior as well as future uses of such defined words and phrases.

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Further, descriptions and disclosures provided in association with one particular embodiment are not limited to that embodiment, and may be applied to any embodiment disclosed herein. Moreover, for the sake of simplicity, the attached figures may not show the various ways in which the disclosed system and apparatus can be used in combination with other systems, and apparatuses.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments. These features and advantages of the embodiments will become more fully apparent from the following description and apportioned claims, or may be learned by the practice of embodiments as set forth hereinafter.

In accordance with the present disclosure, a balloon catheter is disclosed. The balloon catheter of the present invention is used for pre-dilatation of a native stenotic valve prior to implantation of a heart valve. The balloon catheter may also be used for pre-dilatation in any coronary and/or peripheral artery.

The balloon catheter of the present invention includes a tubular member (tubular element) mounted over an outer surface of a balloon (or any other equivalent expandable structure). The tubular member of the present invention is expanded upon expansion of the balloon thereby leading to breakage of calcified tissues at an implantation site.

The tubular member includes three major sections i.e. a proximal section, a middle section and a distal section. The proximal and the distal sections include struts that are anchor shaped and hence, allow better attachment of the tubular member with the balloon. The middle section is provided with a plurality of closed cells having zig-zag elements. The closed cell structure in the middle section allows better expansion of the tubular member once the balloon is inflated. The zig-zag elements also help to increase the length of the tubular member during balloon inflation. The middle section of the tubular member is provided with at least one marker for better radio-opacity of the tubular member while fluoroscopic imaging. The aforesaid tubular member maintains its shape even if it is crimped for a long time and/or undergoes multiple inflation-deflation cycles. Further, the middle section includes a proximal middle portion, a distal middle portion, and an intermediate portion connecting the proximal middle portion and the distal middle portion. The intermediate portion includes a plurality of s-shaped links which provide enhanced flexibility and ease in advancement through aortic arc, thereby avoiding any injury to the vessel.

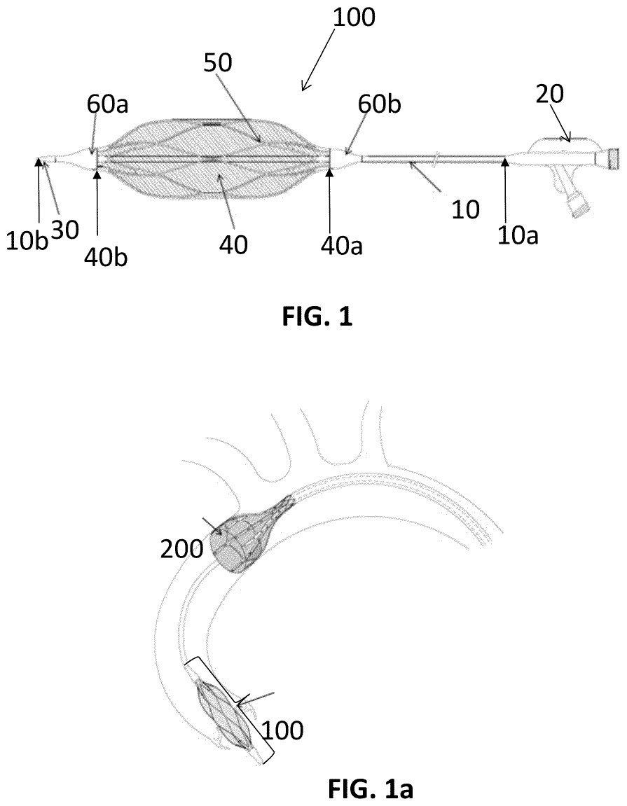

Referring to figures, FIG. 1 depicts the balloon catheter (or valvuloplasty balloon catheter) 100 of the present invention. As mentioned earlier, the balloon catheter 100 of the present invention is used for pre-dilation of a native stenotic valve. The balloon catheter 100 of the present invention may be used to treat stenosis of without limitation, aortic valve, pulmonary valve and bicuspid valve. Moreover, the balloon catheter 100 of the present invention may be used for valve in valve (ViV) procedure and/or for coarctation of aorta before stent placement.

The pre-dilation of the stenotic valve might create debris which when released into the bloodstream may cause blockages in smaller vessels. Hence, the balloon catheter 100 of the present invention may be provided with an embolic filter 200 as shown in FIG. 1a. The embolic filter 200 may be structured to capture as well as remove debris during valvuloplasty procedure. The embolic filter 200 may be a self-expanding filter attached with the balloon catheter 100 at a pre-defined location. In an embodiment, the embolic filter 200 is placed before the aortic arch to avoid any emboli to go further in the bloodstream.

The balloon catheter 100 may include a pre-defined diameter. In an embodiment, the diameter of the balloon catheter 100 is one of, 14 mm, 16 mm, 18 mm, 20 mm, 23 mm or 25 mm. The length of the balloon catheter 100 may be in a range of 1200 mm to 1400 mm. In an embodiment, the length of the balloon catheter 100 is 1300 mm.

The balloon catheter 100 of the present invention includes various components such as, without limitation, one or more of, a catheter tubing 10, a hub 20, a soft tip 30, an inflatable member 40, a tubular member 50 and at least two couplers 60a, 60b.

The catheter tubing 10 of the balloon catheter 100 may be a conventional structure which is utilized to mount the other components of the balloon catheter 100. The catheter tubing 10 may include pre-defined dimensions and shape. The catheter tubing 10 may include a proximal end 10a and a distal end 10b. The proximal end 10a of the catheter tubing 10 may be coupled to the hub 20 of the balloon catheter 100. The distal end 10b of the catheter tubing 10 may be coupled to the soft tip 30.

The hub 20 and the soft tip 30 of the balloon catheter 100 may be conventional structures. The hub 20 may include various ports such as guide wire port, inflation port, etc.

The soft tip 30 may be equivalent to any atraumatic tip known in the art, which is configured to guide the balloon catheter 100 through the body lumen while avoiding any perforation in the body lumen.

The inflatable member 40 for example a balloon, as shown in FIG. 1 is mounted over the catheter tubing 10 towards its distal end 10b. The inflatable member 40 of the balloon catheter 100 may be an expandable structure. The inflatable member 40 may be made of a pre-defined polymeric material having good elasticity and which is capable of enduring high inflation pressures during the valve expansion. The material includes, without limitation, nylon, polyether block amide, polyethene terephthalate (PET), polyamide, polyurethanes, polyvinyl chloride, polyethylene, etc. In an embodiment, the inflatable member 40 is made from polyamide resins.

The inflatable member 40 may be made of a single layer or may include multiple layers. In an embodiment, the inflatable member 40 is a single layered structure. The rated burst pressure of the inflatable member 40 may range from 5 atm to 10 atm. In an embodiment, the rated burst pressure of the inflatable member 40 is 6 atm.

The inflatable member 40 includes a proximal end 40a and a distal end 40b. The distal end 40b of the inflatable member 40 is disposed towards the distal end 10b of the catheter tubing 10. The proximal end 40a is placed opposite to the distal end 40b. The inflatable member 40 tapers towards its ends to connect with the catheter tubing 10 at the proximal end 40a and a distal end 40b as shown in FIG. 1.

Though the present invention is described with the help of a balloon, however, any other equivalent inflatable member 40 capable of being inflated and deflated is also within the scope of the present invention.

The tubular member 50 is attached to or mounted over the catheter tubing 10. The tubular member 50 may be made of a conventional metallic material(s) which includes without limitation, stainless steel, cobalt-chromium, nitinol, etc. In an embodiment of the present invention, the tubular member 50 is made of nitinol owing to its self-expanding and super-elastic properties. The tubular member 50 includes a pre-defined structure which helps to break the calcified tissues. The tubular member 50 may be fabricated using a conventional method. In an embodiment, the tubular member 50 is formed by laser cutting a hollow cylindrical tube.

The inner diameter of the tubular member 50 may range from 2.5 mm to 3.5 mm. In an embodiment, the inner diameter of tubular member 50 is 3.01 mm. The outer diameter of tubular member 50 may range from 3.0 mm to 4.0 mm. In an embodiment, the outer diameter of tubular member 50 is 3.61 mm.

As shown in FIG. 1, the tubular member 50 includes a plurality of closed cells 54. The tubular member 50 may include elongated cells that expand with the inflatable member 40 of the balloon catheter 100.

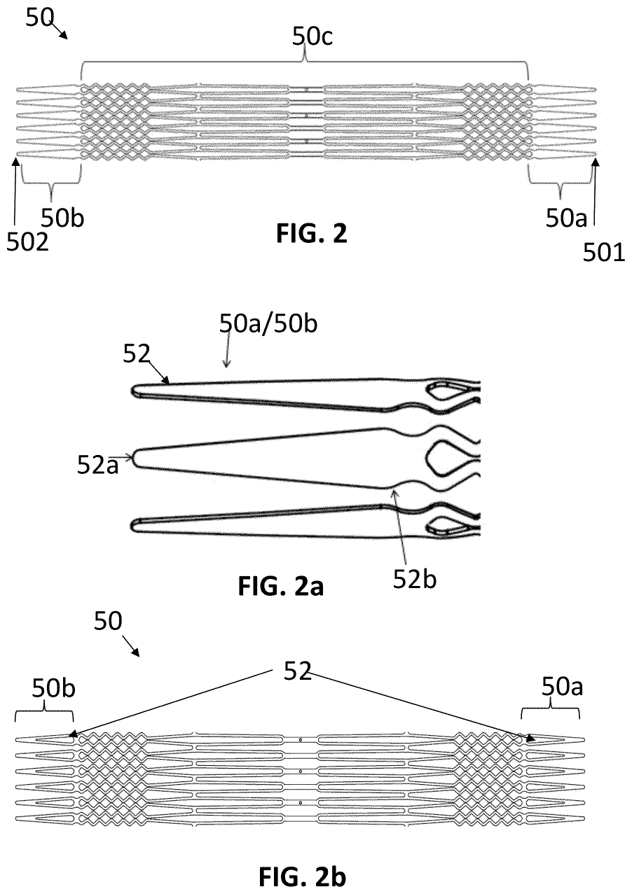

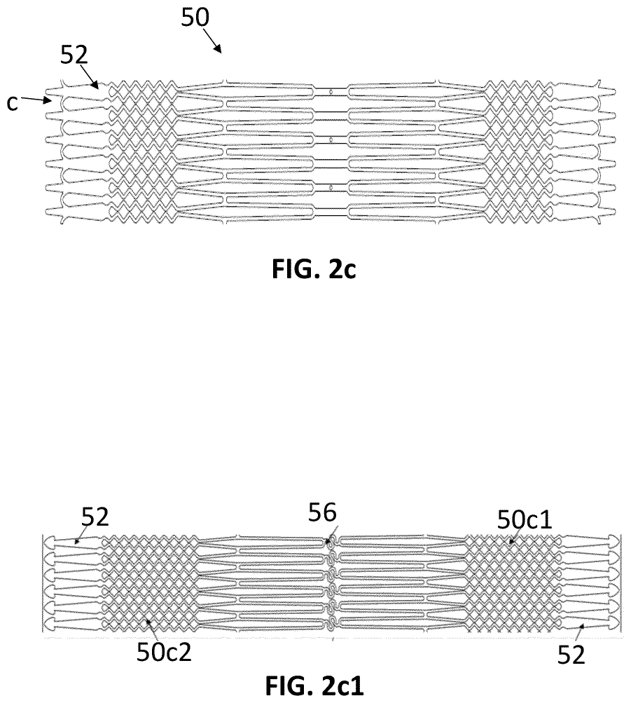

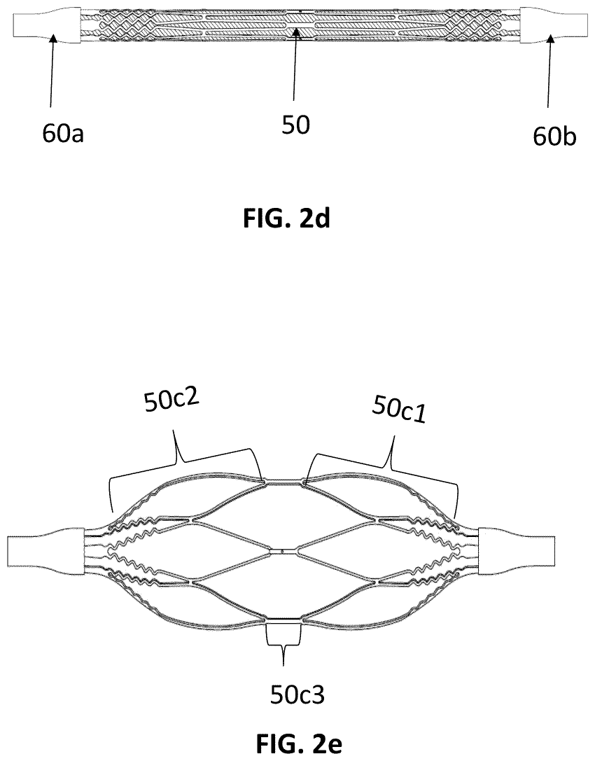

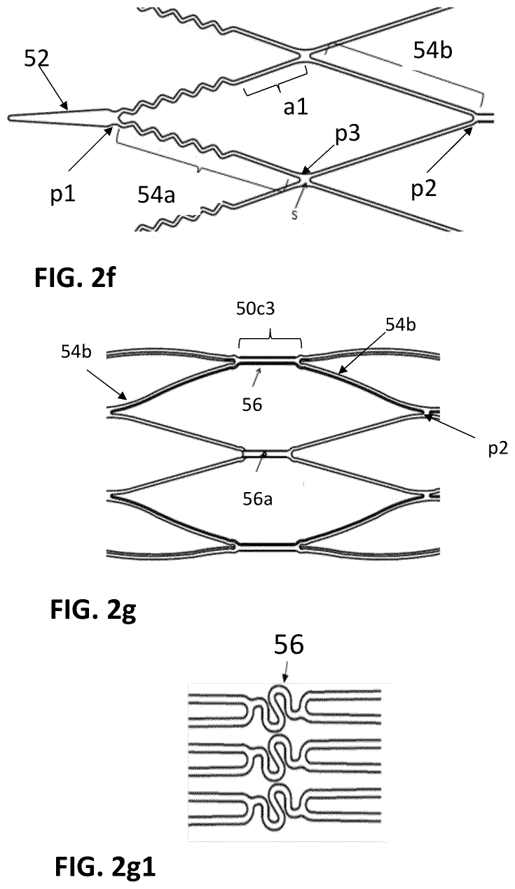

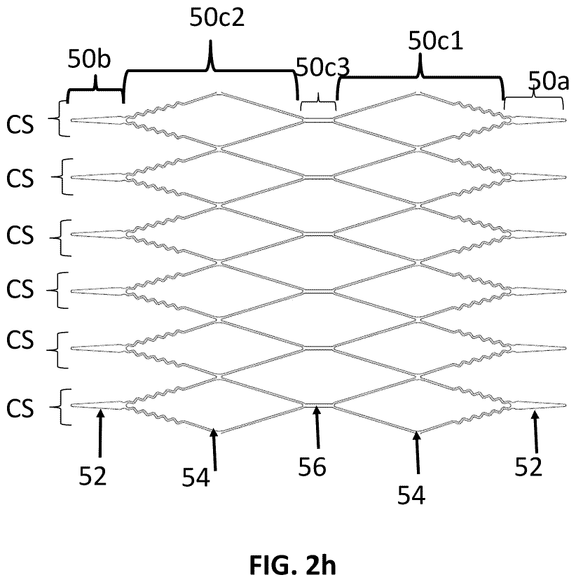

As shown in FIG. 2 more clearly, the tubular member 50 includes a proximal end 501 and a distal end 502. Further, as depicted in FIG. 2, the tubular member 50 includes three sections i.e. a proximal section 50a, a distal section 50b and a middle section 50c disposed there between. The proximal section 50a is disposed towards the proximal end 501 of the tubular member 50 while the distal section 50b is disposed towards the distal end 502. The soft tip 30 is coupled to the distal section 50b of the tubular member 50. The catheter tubing 10 is coupled to the proximal section 50a of the tubular member 50.

The aforesaid coupling of the tubular member 50 of the present invention may be mediated with the help of the couplers 60a, 60b. The couplers 60a, 60b can be for example, sleeves, tubes, rings (O shape, C shape or any other shape), stoppers etc. In an embodiment, the tubular member 50 is coupled via welding technique or combination of welding & adhesive bonding.

The structure of the proximal section 50a and the distal section 50b may be same or different. In an embodiment, the proximal section 50a and the distal section 50b is same. As shown in FIG. 2 and more clearly in FIG. 2a, the proximal section 50a and the distal section 50b include a plurality of first struts 52. The number of first struts 52 may vary depending on the diameter of the inflatable member 40. The number of first struts 52 may range between 02 to 10. In an embodiment, the number of first struts 52 is six.

The first struts 52 may include a pre-defined shape. The shape of the first struts 52 may be any conventional shape. Such shapes may include, without limitation, spear shape, triangular shape, linear leaf shape, lanceolate leaf shape, anchor shape etc. In an exemplary embodiment as depicted in FIG. 2a, the first struts 52 are in the form of spear shaped structures. The said shape of the first struts 52 help to hold the tubular member 50 on to the catheter tubing 10 firmly during the inflation as well as the deflation of the inflatable member 40.

The first struts 52 may be in the form of solid structures as represented in FIG. 2a or may include a cut-out as represented in FIG. 2b. The cut-out structure of the first struts 52 adds extra flexibility and smoothness to the tubular member 50 which in turn helps in its bonding with the soft tip 30/the catheter tubing 10 via the couplers 60a, 60b. Further, the side edges of the first struts 52 may be uniform i.e. straight or non-uniform i.e. having a sine-wave shape, a peak-valley shape, a zig-zag shape, etc.

In another embodiment as depicted in FIG. 2c, each of the first struts 52 is connected to an adjacently placed first strut 52 with the help of a curved connector ‘c’. The utilization of such connectors reduces chance of slippage over the balloon surface.

In yet another embodiment, the first struts 52 are in the form of the anchor shape as depicted in FIG. 2c1. The said shape of the first struts 52 has smooth edges which help to minimize and/or avoid trauma to the vessel wall during retraction and/or expansion of the tubular member 50 post deflation inside the blood vessel. Further, the anchor shape of the first struts 52 imparts strong grip to the couplers 60a and 60b on the distal end 502 and proximal end 501 of the tubular member 50, thereby providing robust attachment of the tubular member 50 on the catheter tubing 10. Strong attachment of the tubular member 50 over the catheter tubing 10 prevents removal of the tubular member 50 during multiple cycles of balloon and inflation and deflation process.

The thickness of the first struts 52 may be in a range of 0.10 mm to 0.50 mm. In an embodiment, the thickness is 0.30 mm.

The first struts 52 include a pre-defined length and width which assist to fix the position of the tubular member 50 within the couplers 60a, 60b (as clearly shown in FIG. 2d) thereby helping the tubular member 50 to withstand high bond strength. The length of the first struts 52 may range from 2 mm to 16 mm. In an embodiment, the length of each first strut 52 is 6 mm-12 mm. The first struts 52 having a length in the aforesaid range enhance the flexibility of the tubular member 50 due to the presence of less material of construction. Also, owing to the use of struts having reduced width, more number of struts is required which in turn increases the radial strength of the tubular member 50.

The middle section 50c extends between the proximal section 50a and the distal section 50b. The total length of the middle section 50c may be in a range of 50 mm to 70 mm. In an embodiment, the total length of the middle section 50c is 62 mm.

As shown in FIG. 2e, the middle section 50c includes three sections i.e. a proximal middle portion 50c1, a distal middle portion 50c2 and an intermediate portion 50c3 connecting the proximal middle portion 50c1 and the distal middle portion 50c2. The proximal middle portion 50c1 is disposed towards the proximal section 50a while the distal middle portion 50c2 is disposed towards the distal section 50b.

The proximal middle portion 50c1 and the distal middle portion 50c2 may contain one or more rows having large cell assemblies. The said cells may either be open cells or closed cells. In an embodiment, the proximal middle portion 50c1 and the distal middle portion 50c2 include a plurality of closed cells having a predefined shape.

The closed cells of the proximal middle portion 50c1 and the distal middle portion 50c2 may include same or differently structured closed cells. As represented in FIG. 2e, the closed cells of the proximal middle portion 50c1 and the distal middle portion 50c2 are same and are referred as closed cells 54. The structure of the closed cell 54 is clearly represented in FIG. 2e/2f.

Each closed cell 54 may include a predefined shape formed by a plurality of struts for example, hexagonal, rhombus, diamond, etc. Each of the closed cells 54 of the present invention includes zig-zag elements. The portion of the closed cell 54 having zig-zag elements is mounted over the tapered portion of the inflatable member 40 in a tapered configuration. The presence of zig-zag shaped elements help to remove the plaque or calcified portion during the expansion of inflatable member 40. The zig-zag elements of the present invention help to adjust the length of the tubular member 50 so that the tubular member 50 smoothly and uniformly expands along with the inflatable member 40.

In an exemplary embodiment of the present invention, the closed cell 54 is a rhombus shaped cell having four struts. In an embodiment shown in FIG. 2f, each closed cell 54 includes a first pair of struts 54a and a second pair of struts 54b. Each first pair of struts 54a is connected to each other at a first peak ‘p1’. The first peak ‘p1’ may be in contact with the first strut 52 and help to connect the first row of the proximal middle portion 50c1 to the first struts 52 of the proximal section/distal section 50a/50b. Each second pair of struts 54b may be connected with each other at a second peak ‘p2’. The second peak ‘p2’ may be in contact with the intermediate portion 50c3 (described below). The first pair of struts 54a may be connected to the second pair of struts 54b at a peak ‘p3’. The peak ‘p3’ allows each closed cell 54 to connect with the adjacent closed cell 54 via a link ‘s’ thereby forming a circumferential row of closed cells 54. The link ‘s’ may be straight link. The length of the links ‘s’ may range from 0.2 mm to 0.8 mm. In an embodiment, the length of each link ‘s’ is 0.47 mm.

The struts of the closed cell 54 (namely, the first pair of struts 54a and/or the second pair of struts 54b) may be identical in structure or may include different structures.

In an embodiment, the first pair of struts 54a and/or the second pair of struts 54b include at least a portion having zig-zag elements. In an embodiment, the first pair of struts 54a and/or the second pair of struts 54b may be entirely zig-zag in shape (not shown). Alternately, each strut of the first pair of struts 54a and/or the second pair of struts 54b may include a zig-zag element and a first element a1 thereby forming a hybrid structure. The first element a1 may include any shape such as without limitation, a straight shape, a spline shape, a semicircular shape, etc. In an embodiment as shown in FIG. 2f, the first element a1 is straight shaped. The length of the zig-zag element and the length of the first element a1 may have a pre-defined ratio. The pre-defined ratio may range between 8:2 to 2:8 respectively for zig-zag element and first element a1 with respect to length. In an embodiment, the pre-defined ratio is 6:4 for zig-zag element and first element a1 respectively.

Each zig-zag element may include a plurality of crests and troughs. The number of crests and troughs may range from two to twelve. In an embodiment, the number of crests and troughs are six. The length of zig-zag element plays an important role in the sizing of the tubular member 50. The length of the zig-zag element may range from 8.5 mm to 10.5 mm. In an embodiment, the length is 9.69 mm. Such length leads to accurate expansion of tubular member 50 when inflated.

The length of the first element a1 may range between 5.0 mm to 7.0 mm. In an embodiment, the length of the first element a1 is 6.16 mm.

Each strut of the second pair of struts 54b may be straight. However, it should be noted that other shapes of such struts are also within the scope of the present invention. Each of the second pair of struts 54b includes a length ranging from 10 mm to 14 mm. In an embodiment, the length is 11.90 mm.

The total length of each closed cell 54 (in compressed stage) may range between 23.5 mm to 32.5 mm. In an embodiment, the total length of each closed cell 54 is 27.75 mm.

The intermediate portion 50c3 includes a plurality of marker links 56 as shown in FIG. 2g. The number of marker links 56 may be dependent upon the number of closed cells 54. The number of marker links 56 may range between 2 to 8. In an embodiment, the intermediate portion 50c3 includes six marker links 56. The marker links 56 help to connect the closed cells 54 of the proximal middle portion 50c1 with the closed cell 54 of the distal middle portion 50c2 via peaks ‘p2’ as depicted in FIG. 2e.

The total length of the marker link 56 may range from 3 mm to 8 mm. In an embodiment, the length is 5 mm. The marker link 56 may include a plurality of markers 56a. In an embodiment, each marker link 56 includes a single marker 56a. The marker 56a may be disposed at the center of marker link 56. The marker 56a may be made of a conventional radiopaque material such as without limitation, platinum, tantalum, platinum tungsten, platinum iridium, gold, etc. In an embodiment, the present invention includes markers 56a made from tantalum and having a round shape. The diameter of the marker 56a may range from 0.25 mm to 0.45 mm. In an embodiment, the diameter of the marker 56a is 0.35 mm.

In another embodiment, the intermediate portion 50c3 includes a s-shaped connecting link 56 as depicted in FIG. 2g1. The presence of the S shaped link in the intermediate portion 50c3 imparts enhanced flexibility, reduces stress at the proximal end 501 and the distal end 502 and helps in withstanding high pressure to the tubular member 50. Moreover, the s-shaped links help in easy advancement of the tubular member 50 through the aortic arc owing to enhanced flexibility and thus avoids vessel injury.

Further, the presence of the S-shaped links 56 helps to increase inflation and deflation cycles of the inflatable member 40 due to uniform distribution of the stress on the tubular member 50. Owing to uniform stress distribution, the tubular member 50 can withstand high pressure without detachment of the couplers 60a and 60b with the catheter tubing 10.

Hence, as per the above exemplary embodiments, the structure of the tubular member 50 is in the form of a plurality of columns ‘CS’ which are replicated radially to form the complete structure of the tubular member 50. Each column includes a first strut 52 of the proximal section 50a, a closed cell 54 of the proximal middle section 50c1, a marker link 56 of the intermediate portion 50c3, a closed cell 54 of the distal middle portion 50c2 and a first strut 52 of the distal section 50b (in the aforesaid sequence). Such columns may be connected with each other at peak (point) ‘p3’ via links ‘s’. The number of columns may vary depending upon the diameter of the inflatable member 40. In an embodiment, the tubular member 50 includes six columns ‘CS’ as represented in FIG. 2h. The above disclosed middle section 50c of the tubular member 50 may reside over the inflatable member 40 and expands along with the inflatable member 40 to break the calcified tissues at the time of pre-dilation. Owing to the presence of larger cells and the aforesaid struts in the middle section 50c, the stresses which act upon the tubular member 50 at the time of inflation-deflation distributes uniformly thereby reducing any chances of breakage of the struts therein.

The design of tubular member 50 help in distributing the forces that are created when the inflatable member 40 of balloon catheter 100 is inflated.

The above disclosed tubular member 50 may be attached with the catheter tubing 10 as shown in FIG. 1 with the help of at least two couplers 60a, 60b. The couplers 60a, 60b may be secured at the distal end 502 and proximal end 501 of tubular member 50. The couplers 60a, 60b may be in the form of polymeric tubes or sleeves. In an embodiment, the couplers 60a, 60b include two bonding sleeves. The couplers 60a, 60b may be secured to the catheter tubing 10 and/or the tubular member 50 via any conventional technique such as without limitation, heating, gluing and welding. In an embodiment, the couplers 60a, 60b are attached via laser welding followed by the adhesive gluing and UV curing. Alternately, the couplers 60a, 60b are bonded with laser welding process to secure the tubular member 50 with the catheter tubing 10 and soft tip 30. The parameters maintained during laser welding are provided as follows:

TABLE 1
Welding Parameter

| Parameters | Value |
|---|---|
| Laser power (W) | 1.3-1.5 |
| Rotation speed (rpm) | 340-360 |
| Number of cycle | 2-4 Times |

The aforesaid bonding provides high tensile strength and bonding strength to the tubular member 50.

In an embodiment, the couplers 60a, 60b include polymeric heat shrink tubes. The tube may be made of without limitation, nylon, polytetrafluoroethylene, polyether block amide, polyolefin, polyurethane, fluorinated ethylene propylene etc. In present invention, polyolefin tube is used for the attaching the tubular member 50.

The outer diameter of the tube may range from 4.0 mm to 9.0 mm. In an embodiment, the outer diameter is 6.23 mm. The inner diameter of tube may range from 3.0 mm to 8.0 mm. In an embodiment, the inner diameter is 5.23 mm.

The foregoing present invention may be explained with the help of below examples:

### Example 1

A fatigue test was performed in order to test performance of the tubular member 50. The fatigue test was performed by subjecting the balloon catheter 100 to rated burst pressure (RBP) by inflation for a predefined period of time. Further, the balloon is deflated and the process is repeated for 20 cycles. The experimental parameters maintained during the test are as follows.

| Parameter | Value | |
|---|---|---|
| Balloon size | 20 × 40 | mm |
| Test pressure time | 15 | seconds |
| Test pressure | 3 | atm |
| Initial pressure | 1 | atm |
| Maximum pressure | 5 | atm |
| Relaxation time between two cycles | 10 | Seconds |

It was observed that the tubular member 50 having straight link 56 detached after 3-5 cycles at a burst pressure of 5 atm. However, there was no detachment observed for the couplers 60a and 60b from the tubular member 50 even after 20 cycles of inflation and deflation even at burst pressure of 5 atm owing to uniform distribution of stress at both the proximal end 501 and distal end 502 of the tubular member 50. Therefore, it provides more robustness to the tubular member 50 during deployment at the implantation site.

### Example 2

A trackability test of the balloon catheter 100 was performed in a three-dimensional model of the artery network consisting of a testing fluid (purified water). The model was fed with a guide wire. Once a predefined temperature is attained, the balloon catheter 100 was inserted into the model over the guide wire at a predefined insertion speed. Further, the force required to navigate the device across the model was calculated.

The experimental parameters maintained during the test are as follows.

| Parameter | Value | |
|---|---|---|
| Balloon size | 20 × 40 | mm |
| Testing fluid temperature | 37 ± 2° | C. |
| Insertion speed | 30 | cm/minute |

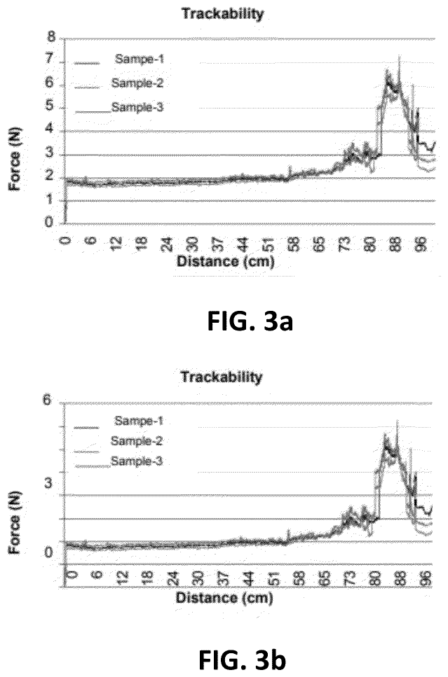

FIG. 3a and FIG. 3b depict a graphical representation of the force required in case of straight link and s-shaped links respectively. It was observed that force require for navigation of the balloon catheter 100 having s-shaped links 56 is two times less as compared to the balloon catheter 100 having the straight link 56.

The scope of the invention is only limited by the appended patent claims. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

We claim:

1. A valvuloplasty balloon catheter (100) comprising:
an inflatable member (40) having anchor shaped ends; and
a tubular member (50) mounted over the inflatable member (40), the tubular member (50) including a proximal end (501) and a distal end (502), the tubular member (50) comprising:
a proximal section (50a) disposed towards the proximal end (501);
a distal section (50b) disposed towards the distal end (502); and
a middle section (50c) disposed between the proximal section (50a) and the distal section (50b), the middle section (50c) including a proximal middle portion (50c1), a distal middle portion (50c2), and an intermediate portion (50c3) connecting the proximal middle portion (50c1) and the distal middle portion (50c2), the proximal middle portion (50c1) and the distal middle portion (50c2) having a plurality of closed cells (54),
wherein at least one of the proximal section (50a) or the distal section (50b) including a plurality of first struts (52), wherein the plurality of the first struts (52) are anchor shaped,
wherein the intermediate portion (50c3) includes a plurality of marker links (56), wherein the marker links (56) are s-shaped.

2. The valvuloplasty balloon catheter (100) as claimed in claim 1, wherein the tubular member (50) is made from, one of, stainless steel, cobalt-chromium or nitinol.

3. The valvuloplasty balloon catheter (100) as claimed in claim 1, wherein the plurality of first struts (52) includes uniform or non-uniform side edges.

4. The valvuloplasty balloon catheter (100) as claimed in claim 1, wherein the plurality of first struts (52) include one of a solid structure or a cut-out.

5. The valvuloplasty balloon catheter (100) as claimed in claim 1, wherein each of the plurality of closed cells (54) is connected to an adjacently placed closed cell (54) other via a link thereby forming a circumferential row of closed cells (54).

6. The valvuloplasty balloon catheter (100) as claimed in claim 1, wherein each of the closed cells (54) includes a rhombus shape formed by a first pair of struts (54a) and a second pair of struts (54b).

7. The valvuloplasty balloon catheter (100) as claimed in claim 6, wherein struts of the first pair of struts (54a) connect with each other and the first strut (52).

8. The valvuloplasty balloon catheter (100) as claimed in claim 6, wherein struts of the second pair of struts (54b) connect with each other and the marker link (56).

9. The valvuloplasty balloon catheter (100) as claimed in claim 6, wherein at least the first pair of struts (54a) and/or the second pair of struts (54b) include one or more zig-zag elements.

10. The valvuloplasty balloon catheter (100) as claimed in claim 6, wherein at least the first pair of struts (54a) and/or the second pair of struts (54b) includes a zig-zag element and a first element (a1) in a ratio ranging from 8:2 to 2:8 forming a hybrid structure.

11. The valvuloplasty balloon catheter (100) as claimed in claim 10, wherein the first element (a1) includes one of a straight shape, a spline shape or a semicircular shape.

12. The valvuloplasty balloon catheter (100) as claimed in claim 1, wherein the tubular member (50) is coupled to a catheter tubing (10) with the help of at least two couplers (60a, 60b).

13. The valvuloplasty balloon catheter (100) as claimed in claim 12 wherein the at least two couplers (60a, 60b) include one of polymeric heat shrink tubes or sleeves.

14. The valvuloplasty balloon catheter (100) as claimed in claim 12, wherein the couplers (60a, 60b) are made from a material selected from nylon, polytetrafluoroethylene, fluorinated ethylene propylene, polyether block amide, polyolefin or polyurethane.
